(19) United States
(12) Patent Application Publication
(10) Pub. No.: US 2011/0076754 A1
(43) Pub. Date: Mar. 31, 2011

(54) DEVICE AND METHOD FOR FILTERING ONE OR MORE PARTICLES TO BE DETECTED FROM A FLUID

(75) Inventors: Alexander Frey, Munchen (DE); Karsten Hiltawsky, Schwerte (DE); Daniel Sickert, Munchen (DE)

(73) Assignee: SIEMENS AKTIENGESELLSCHAFT, Munich (DE)

(21) Appl. No.: 12/893,122

(22) Filed: Sep. 29, 2010

(30) Foreign Application Priority Data
Sep. 30, 2009 (DE) 10 2009 047 802.7

(51) Int. Cl. C12M 1/00; B01D 21/26; B81B 1/00; B81B 7/04

(52) U.S. Cl. 435/283.1; 210/512.1; 210/255

(57) ABSTRACT

A device is disclosed for filtering a particle to be detected from a fluid. In at least one embodiment, the device includes a substrate and, applied above it, a protective layer, a channel being disposed between them for the purpose of receiving a fluid containing different particles. In at least one embodiment, the channel extends at least partially in a spiral shape between its inlet and outlet and on its internal wall contains a coating for attracting a particle type to be filtered, which coating is applied at least on the radially outside subsection of the internal wall of the channel. Owing to the laminar longitudinal movement of the fluid inside the channel a radially outward directed centrifugal force acts on the particles, with the result that per time unit more particles come into contact with the radially outside subsection of the internal wall of the channel than the radially inside subsection of the internal wall of the channel. The filter effect is reinforced as a result.

### PRIORITY STATEMENT

The present application hereby claims priority under 35 U.S.C. §119 on German patent application number DE 10 2009 047 802.7 filed Sep. 30, 2009, the entire contents of which are hereby incorporated herein by reference.

### FIELD

At least one embodiment of the invention generally relates to a device and method for filtering one or more particles requiring to be detected from a fluid.

### BACKGROUND

In order to treat tumor cells it is important to detect what are referred to as circulating tumor cells (CTCs) in the blood. The number of circulating tumor cells in the blood is used to make statements relating to the prognosis of a patient suffering from a metastasized tumor. If the number of circulating tumor cells decreases under therapy, this is an indicator of successful treatment in the sense of a regression of the metastases or, as the case may be, the primary tumor. A great challenge in the study of blood samples stems from the small number of circulating tumor cells. An investigative method must be sufficiently sensitive to detect one tumor cell per milliliter of blood. At the same time the method must be very specific, because one milliliter of blood contains, inter alia, approx. 10 million leukocytes. These share some similar characteristics (e.g. size, cell nucleus) with circulating tumor cells. A fluidic channel is known in which a rod structure (microposts) is introduced. These microposts are coated with EpCAM-AB. Tumor cells having the EpCAM antigen on their cell surface are captured on said surface coating.

WO 2008/130977 (M. Toner) discloses an uncoated microchannel structure which is used for sorting particles. The cells are sorted by exploiting fluidic properties of the particles.

### SUMMARY

In at least one embodiment of the invention, a device and/or method is provided by which particles having a low concentration can be filtered from a fluid.

Advantageous developments of embodiments of the invention are disclosed in the dependent claims.

A fluid is understood to mean a liquid or gas in which there may be found particles and/or particles requiring to be detected and identified, for example environmental pollutants or cancer cells that need to be detected.

A spiral or a spiral-shaped helix is understood to mean a curve that runs around a point or an axis and distances itself from or approaches said point or axis depending on the direction of rotation.

Sorption is understood to mean the formation of a chemical or physical bond between the substance and the surface section. According to this definition sorption includes both absorption and adsorption. In absorption, the substance is absorbed for example by a coating of the resonator that forms the surface section, without forming a phase boundary. The substance is incorporated into the coating. Adsorption, in contrast, results in the forming of a phase boundary.

Particularly conceivable in this case is adsorption in the form of physisorption. The substance attaches itself to the surface section of the resonator as a result of van der Waals or dipole-dipole interactions. Alternatively thereto, adsorption in the form of chemisorption may also take place. In chemisorption, the substance attaches itself to the surface section by forming a chemical bond. The chemical bond is, for example, a covalent bond or a hydrogen bridge bond.

Preferably the sorption takes place reversibly. This means that the substance can also be desorbed (removed) again from the surface section. For example, the substance is detached again by increasing the temperature of the surface section or through the action of a reactive substance. The reactive substance is, for example, an acid or a base with the aid of which the bonds formed during the chemisorption can be broken. This enables the device to be used multiple times. It is, however, also possible for the sorption to be irreversible. In this case the device is used once only as a one-way sensor.

A chemically sensitive layer is understood to mean the exploitation of the aforementioned principles for the purpose of adsorbing a particle type requiring to be filtered.

A chemically sensitive coating is, for example, a polymer film, polystyrene or polymethylacrylate for example. Various substances, for example hydrocarbons, can be adsorbed on the polymer films.

The chemically sensitive coating of the inner surface/internal wall of the channel of the device described hereinbelow also enables particles having an extremely low concentration to be identified in the fluid.

The particles can represent cells, mammalian cells, blood cells, bacteria cells, DNA fragments, beads, virus types, organelles, nanoparticles, molecular complexes, tumor cells, and/or cancer cells. Reliable filtering of individual particle types is possible especially when a multiplicity of different particles having a different concentration are contained in the fluid.

The device for filtering one or more particles from a fluid has a substrate and a protective layer applied above it, a channel being disposed between them for the purpose of receiving a fluid and allowing its laminar flow between the inlet and outlet of the channel 2 in the latter's longitudinal direction, the fluid containing different particles, the channel extending at least partially in a spiral shape between the inlet and outlet.

A coating 5 for attracting a particle type to be filtered 22, 23 is applied at least on the radially outside subsection of the internal wall of the channel. Owing to the laminar longitudinal movement of the fluid inside the channel in the latter's longitudinal direction, a centrifugal force directed radially outward acts on the particles, with the result that per time unit more particles come into contact with the radially outside subsection of the internal wall of the channel than the radially inside subsection of the internal wall of the channel.

Preferably the coating is embodied as a chemically or biochemically sensitive substance to which only a specifically predefined particle type adheres upon contact.

Preferably the particles are cells, mammalian cells, blood cells, bacteria cells, DNA fragments, beads, viruses, organelles, nanoparticles, molecular complexes, tumor cells, and/or cancer cells.

Preferably the chemically sensitive coating chemically or biochemically binds the particle type requiring to be filtered 22, 23, with by preference the aim being to filter tumor cells or cancer cells.

Preferably the average width or cross-sectional area of the channel decreases as a function of the current radius of the spiral, the center point of the radius corresponding to the center of the spiral. A constant centrifugal force is achieved thereby.

Between its inlet and outlet the channel preferably has the shape of a spiral entity whose basic shape is approximated by two sequentially arranged and mutually entangled spirals that are defined by the two polar equations

r=a·θ^{1/m} (1)

and

r=−a·θ^{1/m} (2)

where
θ is the angle variable which, starting from the center point (M5) of the two spirals S3, S4, represents the angular rotation movement traveled by the respective spiral (S3, S4) in the anticlockwise direction.
r(θ) is the resulting distance of the spiral points S3, S4 from its center point as a function of the traveled angular rotation movement θ;
a is a constant on which the distance of the adjacent channels from each other depends;
m is a constant which determines the strength of the dependence of the distance of two adjacent channels from each other on the traveled angular rotation movement θ.

Preferably a plurality of spiral entities are arranged one after another between inlet 3, 30, 300 and outlet 4, 40, 400.

Preferably the inlet and/or outlet of the channel lead/leads out of the substrate vertically to its plane.

Preferably the inlet and/or outlet of the channel lead/leads laterally out of the substrate, by preference on one side.

Preferably the average width or, as the case may be, cross-sectional area of the channel will decrease as a function of the radius/current radius of curvature, as a result of which a constant centrifugal force Fz can be achieved, since the laminar flow velocity of the fluid increases as the cross-section becomes smaller.

Alternatively the inlet and outlet of the channel are brought out of the protective layer of the substrate and/or the protective layer.

A pump is provided to assist the laminar movement of the fluid through the channel. The pump can be disposed on the substrate as a microscale structure or be arranged externally. The pump direction is reversible so that a uniform hydraulic distribution and mixing of the fluid can be realized.

This increases the probability that a particle to be filtered will remain adhered to the chemically sensitive layer. The channel width of the channel is less than 100 micrometers, preferably less than 50 micrometers.

The particles to be filtered have a maximum diameter of between 5 and 20 micrometers. Preferably the radially inside subsection of the internal wall of the channel is not coated with the chemically sensitive coating. This economizes on the expensive material.

The interspace between two channels lying essentially adjacent to each other is less than the width of the channels and is preferably between 5 and 50 micrometers. This enables a compact structure to be realized.

The height of the channel is between 100-times and 1-times the channel width.

Preferably the entire inner surface of the channel is coated with the chemically sensitive material, thereby simplifying the production method.

### BRIEF DESCRIPTION OF THE DRAWINGS

Example embodiments of the invention are explained in more detail in the following figures, in which:

FIGS. 1a, 1b show a device for filtering particles from a fluid in a top view and in schematic cross-section,

FIG. 2 shows a further embodiment variant of the device according to FIG. 1a, 1b with expanded channel geometry,

FIG. 3 shows an embodiment variant of the device from FIG. 1 with modified channel geometry and inlets/outlets in a schematic top view.

FIG. 4 shows an embodiment variant of the device from FIG. 3 with modified channel geometry and inlets/outlets in a schematic top view.

FIG. 5 shows an embodiment variant of the device from FIG. 3 or 4 with expanded channel geometry and inlets/outlets in a schematic top view.

Functionally identical elements of the devices are labeled by the same reference signs in the different figures.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

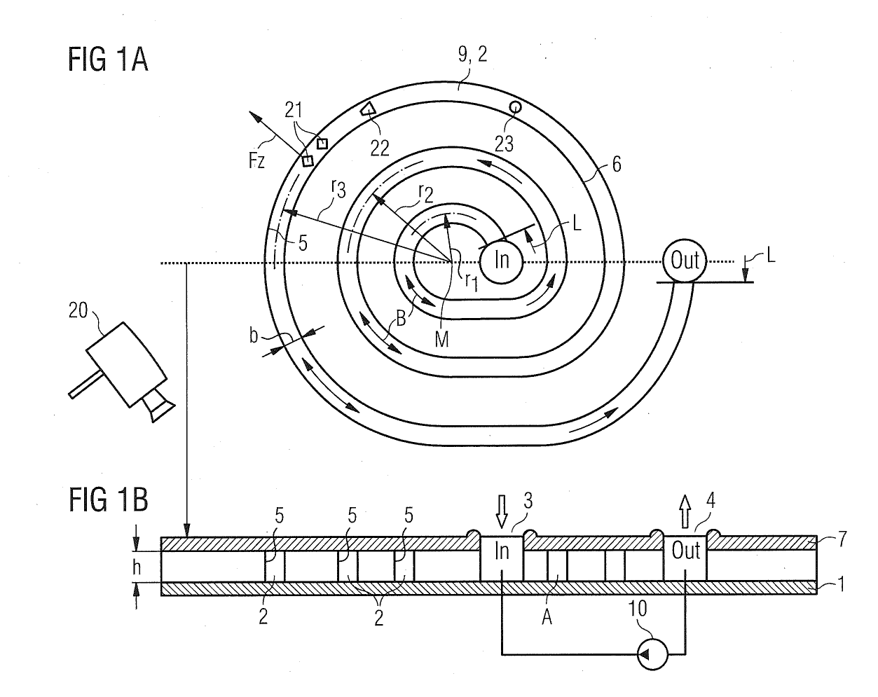

FIGS. 1a and 1b show a device for filtering particles 21, 22, 23 from a fluid 9 in a top view and in schematic cross-section with a substrate 1 and a protective layer 7 disposed essentially parallel thereto, between which a channel 2 for guiding a fluid 9 in its direction of movement B is disposed. Starting from the center M of the substrate 1, the channel 2 runs in a spiral shape in an anticlockwise direction with increasing radius r1, r2, r3 as far as the outside section of the surface area of the substrate 1. For optimization purposes the entire surface area of the substrate 1 will preferably be filled with the channel 2. The inlet 3 to allow the fluid 9 to be supplied to the channel 2 is disposed in the region of the center M of the substrate 1. The outlet 4 is disposed at the other end of the channel 2. The inlet 3 and outlet 4 are in each case brought out vertically with respect to the substrate layer through the protective layer 7. Alternatively the inlet 3 and/or outlet 4 can also be brought out vertically through the substrate 1. The fluid 9 is driven by a pump 10 which is disposed externally or integrated on the substrate 1.

The fluid 9, which may be in liquid or gaseous form, is conducted through the channel 2 in the latter's longitudinal direction B between the inlet 4 and the outlet 5 in a laminar flow.

Alternatively the fluid 9 may be gaseous and contain correspondingly small particles or environmental pollutants that are required to be detected.

Various particles 21, 22, 23 are contained in the fluid 9, one of the particle types 21 or 22 being detected by filtering. At least part of the internal wall 5 of the channel 2 is coated with a chemically sensitive material 5 to which particles to be detected 21 or 22 can adhere when they come into contact with the material 5.

Owing to the spiral-shaped longitudinal movement B of the fluid 9 inside the channel 2 a radially outward directed centrifugal force Fz is exerted on the particles 21, 22, 23 in the fluid 9 which drives the particles 21, 22, 23 onto the radially outside inner surface/internal wall 5 of the channel 2, with the result that per time unit more particles come into contact with the radially outside subsection 5 of the internal wall 5, 6 of the channel 2 than the radially inside subsection 6 of the internal wall 5, 6 of the channel 2.

The number of particles 21, 22, 23 that come into contact with the coated material 5 is dependent, inter alia, on the length L of the channel 2 on the substrate 1, with only the particles requiring to be filtered 22 or 23 remaining adhered to the chemically sensitive material 5. The particles 21, 22, 23 can be, for example, cells, mammalian cells, blood cells, bacteria cells, DNA fragments, beads, viruses, organelles, nanoparticles, molecular complexes, tumor cells, and/or cancer cells. The particles requiring to be detected or, as the case may be, filtered 22, 23 are preferably cancer cells 23 or tumor cells 22 that are contained in low concentration in the fluid 9.

The protective layer 7 and/or the substrate 1 are/is formed from optically transparent material, thereby enabling the filtered particles 22, 23 to be recorded by an optical recording device 20, e.g. a camera or a microscope having optical recognition software, disposed outside the substrate 1 or protective layer 7 and analyzed. This happens already while the fluid 9 is flowing in the channel 2, although it can also take place after the fluid 2 has been emptied from the channel 2. The number of filtered particles 22 or 23 is counted either automatically or manually, thereby enabling the concentration of the particles 22 or 23 in the fluid 9 to be determined.

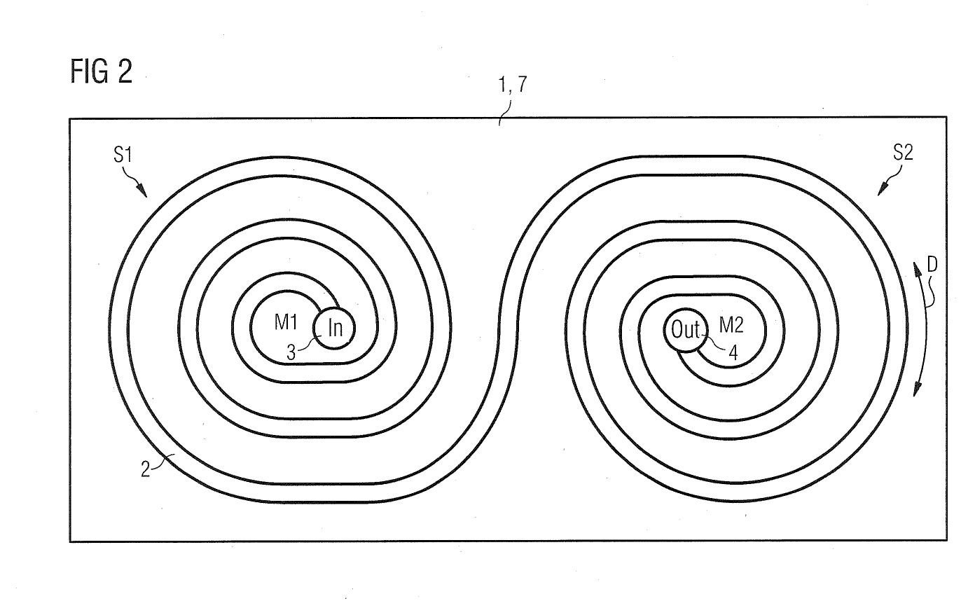

FIG. 2 shows the top view of the device/detection sensor from FIG. 1 with modified geometry of the channel 2.

Between its inlet 3 and outlet 4 the channel 2 in FIG. 2 is shaped into two sequentially arranged and interconnected spirals S1, S2 with mutually opposing direction of rotation D. Located at the center points M1, M2 of the spirals S1, S2 are the inlet 3 and outlet 4, respectively, of the channel 2. Starting from the inlet 3 the first spiral S1 extends in an anticlockwise direction and is followed by the spiral S2, which contracts in the clockwise direction toward the outlet 4 at its center point M2. Alternatively it is also possible for more than two spirals S1, S2 to be arranged sequentially in this way in order to fill out the surface area of the substrate 1 in an optimal manner. In comparison with one large spiral as shown in FIG. 1 a plurality of small spirals S1, S2 connected in series have the advantage that the radii of curvature r1, r2, r3 are smaller on average, as a result of which the average centrifugal force Fz is increased based on the formula

Fz=m*v²/r.

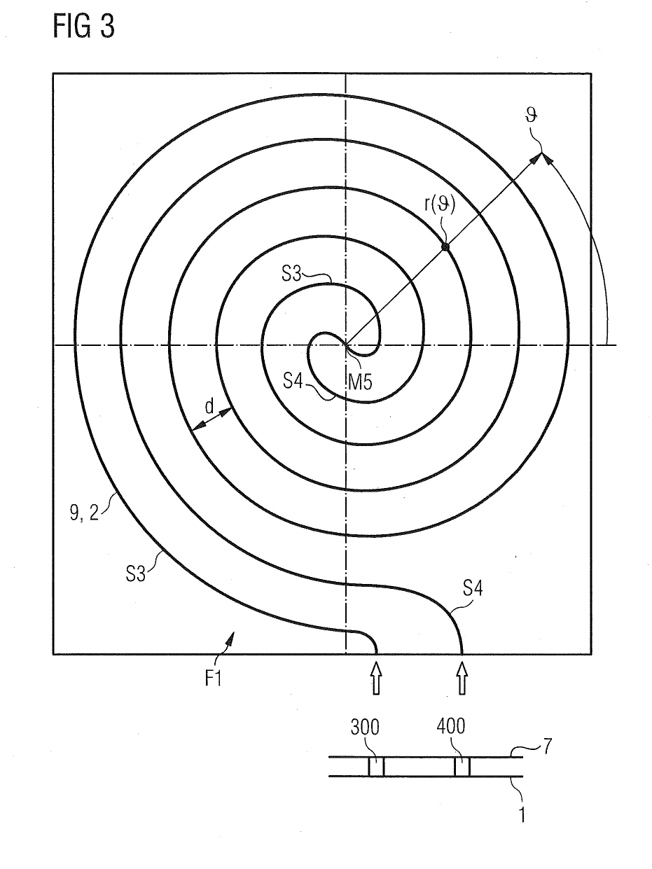

FIG. 3 shows, in a schematic top view, a further embodiment variant of the device from FIG. 1 with modified channel geometry and a modified arrangement and implementation of the inlet 3 and outlet 4.

Between its inlet 3 and outlet 4 the channel 2 has the shape of a spiral entity F1 whose basic shape is approximated by two sequentially arranged and mutually entangled spirals S3, S4 that are defined by the two polar equations

r=a·θ^{1/m} (1)

and

r=−a·θ^{1/m} (2)

where r(θ) is the resulting distance of the spiral points from the center point as a function of the variable θ.
θ is the (angle) variable which, starting from the center point M5 of the two spirals S3, S4, represents the (current) angular rotation movement of the respective spirals S3, S4 in the clockwise direction.
a is a constant on which the distance d of two adjacent channels 2 from each other depends.
m is a constant which determines the strength of the dependence of the distance d of two adjacent channels 2 on the angular movement θ.

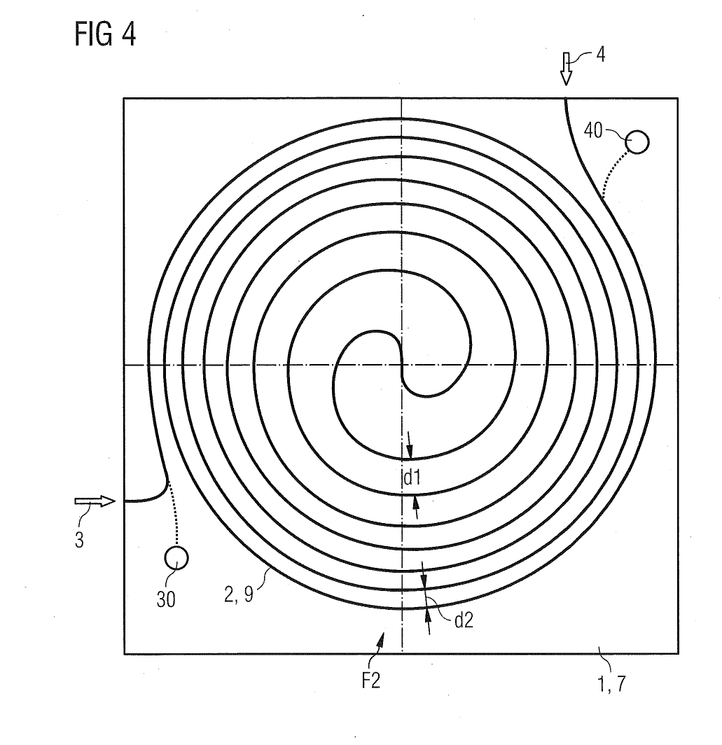

With m=1, one obtains spirals S3, S4 having essentially identical distances d of two adjacent channels 2 from each other independently of the executed angular rotation θ; this spiral shape is depicted in FIG. 3 and is also referred to as Archimedes' spiral. With m=2, the distance d, d1, d2 of two adjacent channels 2 from each other decreases if the absolute value of the executed angular rotation θ increases; this spiral shape is depicted in FIG. 4 with the changes to the distances d1, d2 and is also referred to as Fermat's spiral.

Through embodiment of a spiral entity F1, F2 the respective inlet 3 and outlet 4 of the channel 2 is located outside the spiral entity F1, F2 and not at the center point M of the spiral from FIG. 1. This enables the respective inlet 3 and outlet 4 to be embodied adjacent to each other and/or arranged at any location on the outside of the surface area of the substrate 1 or protective layer 7, as shown for example in FIG. 4 with the inlet 30 and the outlet 40. Preferably the inlet and outlet are brought out of the device laterally between the substrate 1 and the protective layer 7, as shown for example by the in each case laterally brought-out inlet 300 and outlet 400 in FIG. 3. In this embodiment inlet 300 and outlet 400 lie next to each other on one side of the substrate. Bringing the inlet and outlet out laterally allows easy coupling of the channel 2 to external channels of, for example, the pump 10.

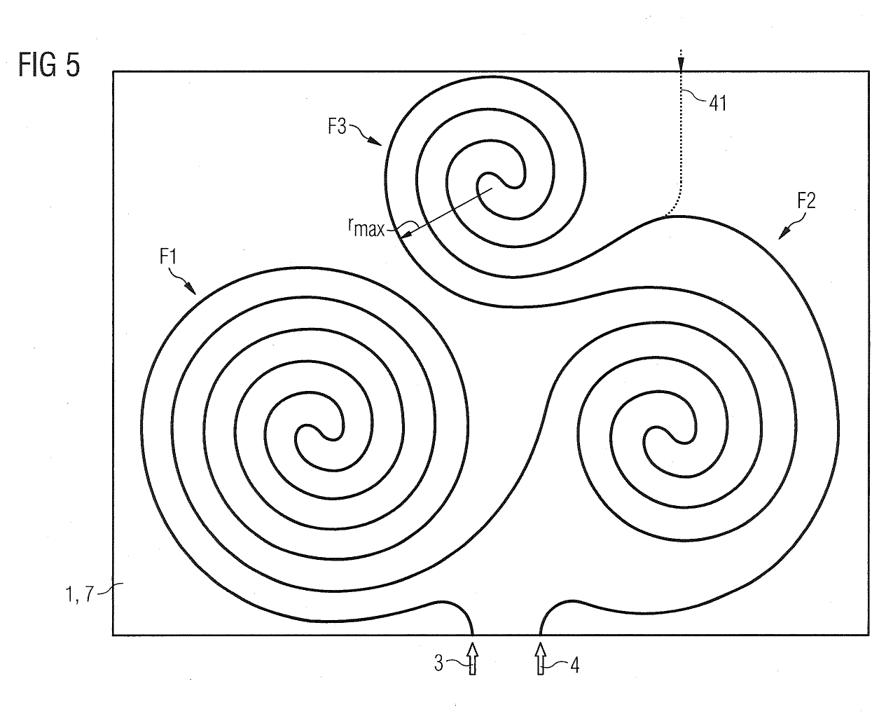

FIG. 5 shows an embodiment variant of the device from FIG. 3 with modified channel geometry in a schematic top view. A plurality of spiral entities F1, F2, F3 from FIG. 3 are arranged in succession, thereby similarly resulting, as in FIG. 3, in smaller average or, as the case may be, maximum radii of curvature r_max, which in turn leads to a higher centrifugal force F acting on the particles 21, 22, 23 in the fluid 9.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A device for filtering one or more particles to be detected from a fluid, the device comprising:
a substrate; and
a protective layer applied above the substrate, a channel being disposed between the substrate and the protective layer for receiving a fluid and allowing its laminar flow between an inlet and an outlet of the channel in a longitudinal direction of the channel, the fluid containing different particles,
wherein the channel extends at least partially in a spiral shape between the inlet and outlet, wherein a coating, for attracting a particle type to be filtered, is applied at least on a radially outside subsection of an internal wall of the channel and wherein, owing to the laminar longitudinal movement of the fluid inside the channel in the longitudinal direction of the channel, a radially outward directed centrifugal force is exerted on the particles, with a result being that, per time unit, more particles come into contact with the radially outside subsection of the internal wall of the channel than a radially inside subsection of the internal wall of the channel.

2. The device as claimed in claim 1, wherein the ratio of a maximum diameter of the particle to be filtered to a maximum width of the channel is less than 0.1.

3. The device as claimed in claim 1, wherein the coating is embodied as a chemically sensitive substance to which only a specifically defined particle type adheres upon contact.

4. The device as claimed in claim 1, wherein the particles are at least one of cells, mammalian cells, blood cells, bacteria cells, DNA fragments, beads, viruses, organelles, nanoparticles, molecular complexes, tumor cells, and cancer cells.

5. The device as claimed in claim 1, wherein the chemically sensitive coating chemically or biochemically binds the particle type to be filtered to itself, and wherein, by preference, an aim is to filter tumor cells or cancer cells.

6. The device as claimed in claim 1, wherein an average width or the cross-sectional area of the channel decreases as a function of the radius, a center point of the radius corresponding to the center point of the spiral.

7. The device as claimed in claim 1, wherein between the inlet and the outlet, the channel includes a shape of a spiral entity whose basic shape is approximated by two sequentially arranged and mutually entangled spirals that are defined by the two polar equations
r=a·θ^{1/m} (1)
and
r=−a·θ^{1/m} (2)
where
θ is the angle variable which, starting from the center point of the two spirals, represents an angular rotation movement of the respective spiral in an anticlockwise direction.
(θ) is a resulting distance of the spiral points from its center point as a function of the traveled angular rotation movement θ;
a is a constant on which a distance of adjacent channels from each other depends; and
m is a constant which determines a strength of dependence of the distance of two adjacent channels from each other on the traveled angular rotation movement θ.

8. The device as claimed in claim 1, wherein a plurality of spiral entities are arranged in succession between the inlet and the outlet.

9. The device as claimed in claim 1, wherein the inlet and the outlet of the channel lead out of the substrate vertically with respect to its plane.

10. The device as claimed in claim 1, wherein at least one of the inlet and the outlet of the channel lead laterally out of the substrate, preferably on one side.

11. The device as claimed in claim 1, wherein at least one of the substrates or layers delimiting the channel includes optically transparent material, thereby enabling the particles fixed by the coating to be recorded by way of an optical recording device.

12. The device as claimed in claim 1, wherein the substrate layer or the protective layer is removable in order to allow the particles fixed by the chemically sensitive layer to be recorded by way of an optical recording device.

13. The device as claimed in claim 1, further comprising:
a pump to assist the laminar movement of the fluid through the channel, disposed integrated on the substrate or arranged externally, the pump direction being reversible to provide more uniform utilization of the chemically sensitive layer.

14. The device as claimed in claim 1, wherein a volume of the fluid to be processed is less than or equal to 5 ml.

15. The device as claimed in claim 1, wherein the coating consists of antigens which can bind precisely one cell shape to a defined antibody.

16. The device as claimed in claim 1, wherein the channel width is less than 100 μm.

17. The device as claimed in claim 1, wherein the particles to be filtered include a maximum diameter of between 5 and 20 μm.

18. The device as claimed in claim 1, wherein the radially inside subsection of the internal wall is uncoated or coated with a chemically insensitive layer.

19. The device as claimed in claim 1, wherein an interspace between two channels lying essentially adjacent to each other is less than the width of the channels and is preferably between 5 μm and 50 μm.

20. The device as claimed in claim 1, wherein a height of the channel is between 100-times and 1-times the channel width.

21. The device as claimed in claim 1, wherein essentially the entire inner surface of the channel is coated with the chemically sensitive material.

22. The device as claimed in claim 1, wherein the channel length between inlet and outlet is so long that the coating has been touched once, ten times, a hundred times or a thousand times as often by particles as the number of particles.

23. The device as claimed in claim 1, wherein a plurality of substrates are cascaded at least one of next to one another and on top of one another, the channels of the individual substrates being connected to one another.

24. The device as claimed in claim 2, wherein the coating is embodied as a chemically sensitive substance to which only a specifically defined particle type adheres upon contact.

25. The device as claimed in claim 10, wherein at least one of the inlet and the outlet of the channel lead laterally out of the substrate on one side.

26. The device as claimed in claim 16, wherein the channel width is less than 50 μm.

27. The device as claimed in claim 19, wherein an interspace between two channels lying essentially adjacent to each other is between 5 μm and 50 μm.
